(19) United States
(12) Patent Application Publication
(10) Pub. No.: US 2020/0315520 A1
(43) Pub. Date: Oct. 8, 2020

(54) Physiological method to regulate Immune and Endocrinal systems to cure Cancers, blood and neurological diseases.

(71) Applicant: Mounira Mohamad Kaafarani, Beirut (LB)

(72) Inventor: Mounira Mohamad Kaafarani, Beirut (LB)

(21) Appl. No.: 16/374,394

(22) Filed: Apr. 3, 2019

(51) Int. Cl. A61B 5/00; A61M 16/04

(52) U.S. Cl. CPC A61B 5/4227; A61M 16/0402; C12N 5/0635; A61M 2210/0656; A61B 5/4277

(57) ABSTRACT

The Invention of a method of Closing an Orifice located right behind the Epiglottis under the vocal cords unlocks the keys of Immune System and adjusts the Endocrine system. This Orifice is responsible of letting Saliva streaming abnormally into capillaries to imbue them with extra water and inhibiting digested nutrients of Plasma to enter capillaries due to deregulation of Starling Forces. A phenomenon that damages capillaries membranes by creating perforations and reducing the functioning capacity of the lymphatic system and T, B and NK Cells, responsible of enhancing the Immune System. Closing this Orifice will drain out Extra water (Saliva) of capillaries and regulate Starling forces to enable nutrients to enter bloodstream and to increase cells Metabolism, regulate hormones secretion, enable the maturation of NK cells to kill cancers, regulate blood sugar, regulate blood pressure, repair brain's neurons and reproduce new cells to replace defected ones.

### TECHNICAL FIELD

This Invention relates to a physiological discovery. To be more specific it uncovers the role of an orifice located in human mouth, right behind Epiglottis underneath vocal cords that is responsible of deregulation/regulation of Immune and Endocrine Systems in Human body.

### BACKGROUND ART

There is known in anatomy and physiology of human mouth that Epiglottis located on the root of the tongue and behind it there are the vocal cords at the entrance of the larynx and it describes how Epiglottis block the entrance of larynx while swallowing food. However, the physiological description of this area doesn't mention any role to the passage of Saliva and some other substances stock in this orifice while Epiglottis is in upper position that enters through this orifice to the bloodstream while swallowing to damage capillaries membranes, deregulate Starling forces, weaken the Immune system and to perturb the Endocrine system.

### SUMMARY OF THE INVENTION

An object of the present Invention is to uncover the use of the orifice located behind Epiglottis to identify all chemicals and hormones responsible of deregulating Immune and Endocrine systems.

In accordance with the present Invention, closing this orifice behind Epiglottis will stop Saliva and nutritional substances that are stock in this area from going directly in the bloodstream to disturb its water and Nutritional composition by deregulating the intra and extra starling forces and damaging the capillaries membranes.

According to this Invention, stopping Saliva from streaming directly in blood circulation will regulate chemicals and nutritional composition inside and outside capillaries, regulating starling forces and protecting capillaries membranes from damages. It also protects the inner of each cell from damages by providing the blood capillaries with all water and nutrients needed for the metabolism in every cell. Thus, it regulates the sugar level in the blood and the blood pressure inside and outside capillaries.

Further, according to the present Invention, as the Blood composition and circulation are regulated, the Endocrine system will behave accordingly to regulate hormones released in the bloodstream, thereby controlling the whole neurological system disorders.

### BRIEF DESCRIPTION OF THE DRAWINGS

FIG. 1 is a cross-sectional view of the orifice 1 behind Epiglottis 2 between the ventricle of Larynx 5 and the vocal cords 4.

FIG. 2 is a profile view of the location of the orifice 1 right behind Epiglottis 3 under the vocal cords 2 where the thyroid 4.

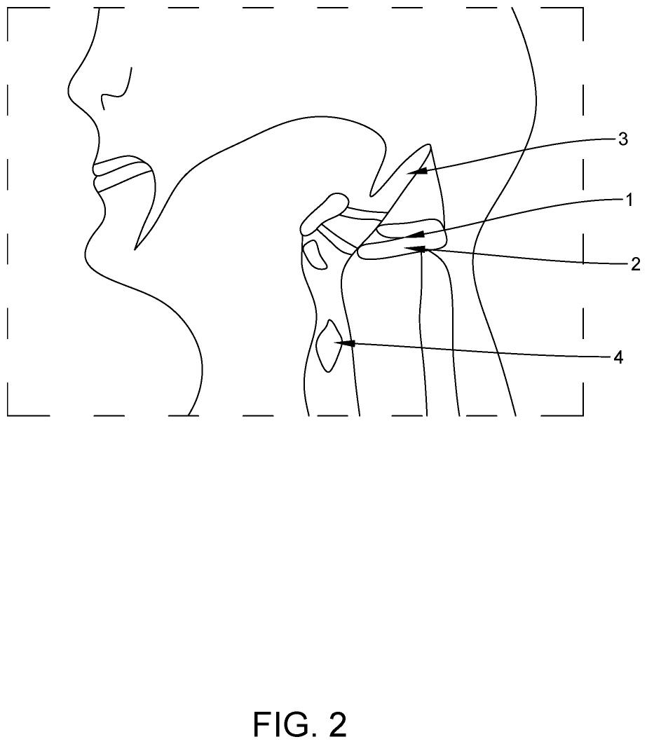

FIG. 3 is a transversal section view of the orifice and the arrow is where the orifice located.

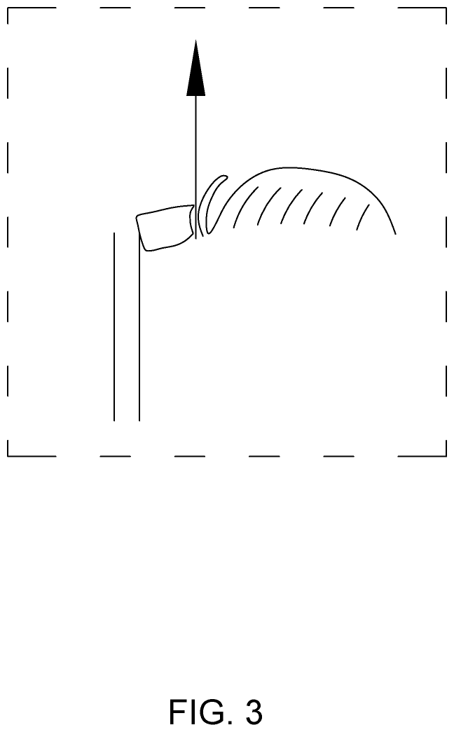

FIG. 4 is a view of the main glands, Hypothalamuses, Thyroids and Pituitary Gland that are mainly disturbed by the opening of this orifice.

### DETAILED DESCRIPTION OF THE INVENTION

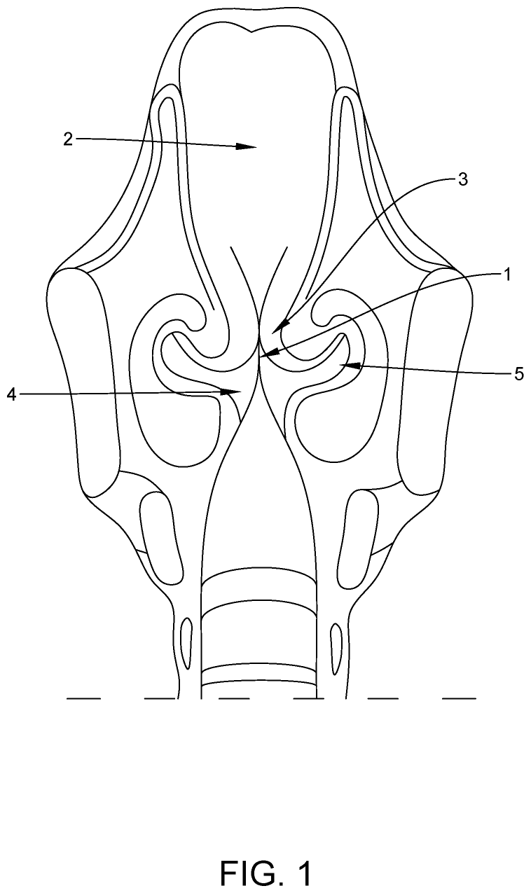

An Orifice 1 in FIG 1. located on the root of the tongue, right behind Epiglottis 2 and before the Larynx 5 in Human Mouth that permeates the infiltration of Saliva while swallowing food which will be abnormally absorbed in the blood circulation leading to the deregulation of its nutritional composition. The abnormal absorption of Saliva will saturate the Blood capillaries with extra water and substances with different chemical composition of the nutritional elements typically absorbed by the blood through the Intestinal tissues. The Saturation of capillaries with extra-water will deregulate Starling forces intra and extra vascular system and will force out of capillaries the extra water of Saliva (99.5 of its composition is water).

Saliva has a similar composition of blood Plasma with differences in the coefficients of their components. But unlike Blood nutrients and chemical composition, Saliva composition cannot be recognized and absorbed normally by Human cells. Lacking the right digested nutrients and chemical enzymes to be absorbed by each cell will decrease its metabolism, a mechanism that will increase the sugar level in the blood and decrease the energy manufacturing inside each cell creating diabetes.

The deregulation of Starling Forces intra and Extra vascular system will in turn deregulate the Lymphatic System, where T, B and Natural Killer cells (NK) are responsible for Human Immune System. The absorption of Immature nutrients inside the lymphatic nodes is responsible of blocking T, B and NK cells from defending body against diseases and of weakening of the Immune System, thus increasing the probability of human immunodeficiency virus infection and over time Acquire Immunodeficiency Syndrome AIDS.

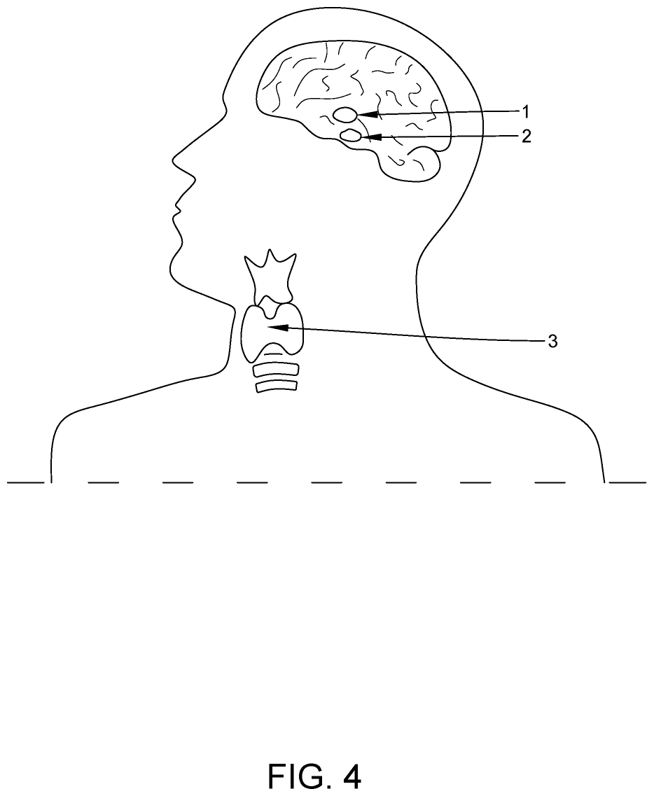

Hypothalamuses, Pituitary Gland, Thyroid in FIG. 4 and Thymus Glands are dramatically affected by Blood disorder and Immune System deregulations. The disturbed cells functioning and metabolism will increase the deficiency in different organs. Therefore, the signalization system is perturbed and the hormones secretion is perturbed as well, leading to perturbations in the Neurological System and over time to neurological diseases as Parkinson's.

Moreover, the Neurological disorders caused by the dysfunction of endocrine system will be the cause of the brain electrical and sensations disorders that cause Epilepsy. Therefore, the purification of the bloodstream of extra water, nutrients and chemicals will regulate hormones released in the bloodstream system to regulate in turn the electrical signalization in the brain.

The location of this Orifice right behind Epiglottis surrounded by mandibular muscles, Jugular muscles and its contraction by every movement like talking, eating, swallowing and stressing out, make this orifice opened to let saliva and other micro-nutrients existing in the mouth stream inside the blood without passing by its normal decomposition through the intestinal absorption system. The increasing quantity of Saliva passing across this Orifice will be critical to the health condition and will cause tumors in human body. As Saliva has the same composition of the blood and it is streaming outside capillaries, it is capable of creating cells in any place where undigested nutrients are accumulated as parasites in any organs to develop cancers.

The normal daily Human activities does not mostly affect the opening of this Orifice. The major reason for its opening is Stress which put a heavy pressure on the upper and lower mouth muscles to let pass more Saliva across this Orifice leading to the deregulations explained above thus the association between stress and deficiency of the Immune system and other physiological diseases in Human body and the association between stress and cancers.

Purifying the bloodstream from extra water and other substances abnormally absorbed in the bloodstream will eliminate the cause of Vitiligo that depigments the skin color.

To cure cancers, diabetes, Parkinson's, Epilepsy and Vitiligo, the regulation of the Immune system and Endocrine system are the target. Enabling the Innate Immune system is by bringing to maturity T, B and NK cells that will make the marvelous Job that our Mother Nature has assigned to our Body in order to repair itself. Thus, by closing this orifice we identify the substances responsible of bringing Immune Cells to Maturity, Chemical enzymes and Nutrients components that can be industrially reproduced in medicinal pills. This Invention can identify the keys of dysfunction of Immune and Endocrine systems and the causes of their deregulations and it identifies the key components of their regulation by closing this orifice. The Industrial use of this invention will be by reproducing the substances identified responsible of curing cancers, Epilepsy, Parkinson's, diabetes and Vitiligo in medicinal pills to be prescribed to patients.

1- The Invention of a method of Closing an Orifice located right behind the Epiglottis under the vocal cords that unlocks the keys of Immune System and adjusts the Endocrine system,
Characterized in that said closing orifice curing cancers by letting the body Immune system releasing a specific substance or substances that extract the tumor cells, stop their proliferation and reduce tumor size.

2- The Orifice as claimed in claim 1 characterized in that regulating blood sugar level by reducing the amount of water inside cells to adjust the intra and extra vascular starling forces and to let enough nutrients and matured enzymes to enter each cell to increase metabolism and to regulate blood sugar level to cure diabetes.

3- The Orifice as claimed in claim 1 characterized in that eliminating extra water and other micro substances contained in human saliva while absorbed abnormally and directly in bloodstream that causes vitiligo by purifying blood from strange substances and regulating skin tissues functioning and pigmentation cells.

4- The Orifice as claimed in claim 1 characterized in that curing Epilepsy by regulating brain's neurons electrical signals and regulating hormones released in bloodstream to strengthen neurological system.

5- The Orifice as claimed in claim 1 characterized in that curing Parkinson's by regulating hormones secretion of Dopamine, reproduction of brain's neurons and repairing bodies nerfs to strengthen their connection to neurological system.

6- The Orifice as claimed in claim 1 characterized in that curing Alzheimer by reproduction of brain neurons and repairing the existing neurons by adjusting hormonal and nutritional elements that enter each cell.

7- The Orifice as claimed in claim 1 characterized in that rejuvenating facial cells and retarding aging process by releasing substances and hormones in the bloodstream to regulate cells chemical substances.

8- The Orifice as claimed in claim 1 characterized in that curing Influenza caused by cold weather and/or stressful situation that causes the opening of this orifice when mouth muscles are contracted and letting a big amount of saliva to stream in the blood.

9- The Orifice as claimed in claim 1 characterized in that strengthening Body Immune System and cure persons attained with AIDS by acting on the maturation of T, B and Natural Killer cells of the Immune System to eliminate AIDS symptoms.

10- The Orifice as claimed in claim 1 characterized in that correcting heart functioning and reducing heart diseases by correcting Blood pressure to relief pressure from heart muscles by regulating Intra and extra vascular Starling Forces.

11- The Orifice as claimed in claim 1 characterized in that correcting Kidney function by draining extra water out of capillaries, water that come in the blood in abnormal amount of Saliva and blocks capillaries from absorbing Nutrients needed for cells metabolism, deregulating the lymphatic system and the kidney function.

12- The Orifice as claimed in claim 1 characterized in that correcting Liver function by draining Extra water out of capillaries that inhibits capillaries from absorbing all nutrients needed and causing abnormal filtration of extra water and nutrients in the interstitial liquid leading to swelling of the lymphatic system and the perturbation of the endocrine system.

13- The Orifice as claimed in claim 1 characterized in that regulating Blood Pressure by regulating Starling forces on the level of capillaries.
